(19) United States
(12) Patent Application Publication
(10) Pub. No.: US 2006/0058767 A1
(43) Pub. Date: Mar. 16, 2006

(54) Diapers with improved leak prevention

(76) Inventors: Fen Zhang, San Francisco, CA (US); Wen Shi, San Francisco, CA (US)

Correspondence Address: PERKINS COIE LLP, P.O. BOX 2168, MENLO PARK, CA, 94026, US

(21) Appl. No.: 10/990,691

(22) Filed: Nov. 16, 2004

Related U.S. Application Data
(63) Continuation-in-part of application No. 10/941,490, filed on Sep. 14, 2004.

(51) Int. Cl. A61F 13/15

(52) U.S. Cl. 604/385.24; 604/385.31

(57) ABSTRACT

The present invention provides diapers with improved leak prevention, especially around the waist of the wearer. Diapers according to the invention may have a guard, for example, a leak-proof guard, to contain what is inside the diaper and thus prevent leaks that could occur without the guard. Diapers according to the invention provide added leak protection particularly on the front and backside of the baby.

This application is a continuation-in-part of U.S. application Ser. No. 10/941,490, filed Sep. 14, 2004, which is incorporated herein by reference.

### BACKGROUND

Diapers are used to protect and comfort babies and infants, who may wear diapers at various times of day and night and for extended periods of time. Diapers are also often worn on travel or outside the home where switching a diaper is difficult to perform. Diapers may be disposable or reusable and they can be designed in different ways depending on the size of the baby, the baby's gender, the particular use of the diaper contemplated, and other considerations like medical conditions, including skin conditions, or the preferences of the baby's parents.

As babies have typically not yet acquired the capacity to control their excretion, they may deposit fluids and other matter into the diaper at any time. Once a baby excretes into the diaper, it is desirable to contain the fluids and other matters inside the diaper and to prevent their leakage to the outside of the diaper or to places inside the diaper that are not contemplated to be so accessible. Containing a baby's excrements inside a diaper can be challenging, in particular when a baby is breast-fed which often results in fluid or watery feces. Also, a baby may excrete at times in a sudden or abrupt manner so that leak prevention is particularly difficult.

Attempts to prevent leakage from baby diapers have been made, but with insufficient success. Preventing leaks from a baby's diaper can be challenging because the baby is very fragile and therefore requires that the diaper is gentle and flexible. However, the more gentle and flexible the diaper is, the more likely leaks will occur. On the other hand, a firm diaper that's best suited to prevent leaks may be detrimental to the baby, especially when worn for longer periods. Designs for diapers are needed that can effectively prevent leakage while friendly to the baby for desired periods of time. The present invention provides such designs for diapers.

### SUMMARY OF THE INVENTION

A diaper according to the current invention provides improved leak prevention. Diapers according to the invention provide in certain embodiments improved leak prevention around the waist of the baby, and, in other certain embodiments, on the front and backside of the baby or only on the front or only on the backside of the baby. In certain embodiments, a diaper according to the invention comprises a leak-proof guard capable of providing improved leak prevention. A leak-proof guard according to certain embodiments of the invention provides added leak protection around the waist of the baby and preferably on the front and backside of the baby and most preferably on the backside of the baby. In certain preferred embodiments, the leak-proof guard of the invention improves leak prevention in the area of the spinal column of the baby.

A leak-proof guard of the invention, in certain embodiments, is long enough to fit around the waist of the baby, in certain other embodiments, it covers a fraction of the waistline of the baby. The width of the leak-proof guard can be measured as the extension of the leak-proof guard of the invention along an axis perpendicular to the waistline of the baby and parallel to the baby's skin. In certain embodiments, the width of the leak-proof guard is uniform or substantially uniform, while in certain other embodiments, the width of the leak-proof guard may vary. In certain embodiments, the width of the leak-proof guard is sufficient to improve leak prevention of the diaper but not so much as to unduly interfere with the comfort and/or health of the baby. A leak-proof guard with varying width, in certain embodiments, has a minimum width and a maximum width. The minimum width of the leak-proof guard, in certain embodiments, is sufficient to achieve a desired improvement in leak-prevention of the diaper.

A leak-proof guard according to certain embodiments of the invention comprises a band, a pad, a cushion, a pillow, a support, an absorber, a roll, or other device capable of making the diaper less likely to leak when compared to a diaper without the leak-proof guard. A leak-proof guard of the invention may, in certain embodiments, be comprised of a single band, pad, cushion, pillow, support, absorber, roll, or other device, or it may be comprised of more than one band, pad, cushion, pillow, support, absorber, roll, or other device. The leak-proof guard of the invention, in certain embodiments, may be in the shape of a band, a line, a dot, a square, a rectangle, a circle, an arc, a diamond, a trapezoid, or any other shape that can be arranged in the diaper so that the leak-proof properties of the diaper are improved. The orientation of the leak-proof guard of the invention in the diaper, in certain embodiments, can be parallel to the waist line of the diaper, or at an angle to the waist line, when measured between the waist line of the diaper and the longitudinal axis of the leak-proof guard, if one exists.

A leak-proof guard of the invention, in certain embodiments, can be made of different materials, for example, gauze, cloth, paper, sponge, plastic, or any other material that is capable of improving leak prevention and that does not unduly interfere with the baby's comfort and/or health. The thickness of the leak-proof guard may vary between embodiments and is such that it improves the leak prevention of the diaper but does not unduly interfere with the comfort and/or health of the baby.

In certain other embodiments, a leak-proof guard of the invention comprises a sealing zone, for example, with an elastic zone. The elastic force of the zone, in certain embodiments, is created through an elastic band or any other device capable of creating an elastic force. The extent of the elastic force, in certain embodiments, is sufficient to improve the leak prevention properties of the diaper but not so high as to unduly interfere with the comfort and/or health of the baby. In certain other embodiments, the leak-proof guard is a pad on the waistband of the diaper.

In certain embodiments, a diaper comprises more than one leak-proof guard of the invention and the leak-proof guards may be designed in the same way or in different ways.

### BRIEF DESCRIPTION OF THE DRAWINGS

FIG. 1. An example of a diaper is shown. The diaper shown in this example is composed of a main body with a tri-layer structure including a waistband with a waist fastener at each end and seals around the legs of the baby. The waistband is an extension of the waist fasteners and it may make contact with the skin of the baby wearing the diaper. The backside of a diaper like the one shown is most prone to leakage and in particular around the spinal cord region.

FIG. 2. A drawing of a baby's waist and surrounding anatomy is shown. Babies typically have a bulged abdomen and their waist is shaped like a cocktail glass with a larger dimension at the waist which tapes down towards the hip and pelvis.

FIG. 3. A drawing of a baby's waist and surrounding anatomy is shown after wearing a tightly fastened diaper. The baby's abdomen is deformed by the tightened waistband.

FIG. 4. A diaper with an elastic marginal zone is shown that has a lower free end and that is attached to or a part of the diaper at its upper end. The elastic marginal zone extends below the waistband or the structure of the diaper that performs as, or most closely like, a waistband. In certain embodiments, the elastic marginal zone may be an extension of the waistband.

FIG. 5. An example of a leak-proof guard of the invention is shown. In this embodiment, the guard comprises a sealing zone inserted into the diaper. The sealing zone comprises material, as shown in this example as a strip of material, that is attached to the diaper at its upper end, or the attached zone. In a preferred embodiment, the attached upper end of the sealing zone has no or substantially no elastic element and will not wrinkle the main body of the diaper. This will improve the sealing qualities of the leak-proof guard, especially during movement of the main body of the diaper. The sealing zone is not attached to the diaper at its lower end and therefore the lower end is free and opens up a space between the band and the diaper in which the baby's excrement can get caught whereby leakage is prevented. The lower end of the sealing zone preferably has an elastic marginal zone and creates a seal between the sealing zone and the baby's skin.

FIG. 6. The drawing shows a representation of a baby wearing a diaper with a leak-proof guard of the invention. In this example, the guard comprises an elastic band that, as a result of the snuggly fit of the leak-proof guard of the invention comprising an elastic band and the loosely fitting main body of the diaper, creates a reservoir to temporally store feces during a bowl movement of the baby and reduces the incident of diaper accident.

FIG. 7. The drawing shows a diaper with a leak-proof guard of the invention. In this example, a soft pad is incorporated in the waistband that helps to strengthen the seal around spinal region.

FIG. 8. A diaper with a mini-skirt leak-proof guard according to certain embodiments of the invention. Example 1 provides more detail on this embodiment.

FIG. 9. A diaper with a mini-skirt leak-proof guard with soft foam insert according to certain embodiments of the invention. Example 2 provides more detail on this embodiment.

FIG. 10. A diaper with a soft pad type leak-proof guard according to certain embodiments of the invention. Example 3 provides more detail on this embodiment.

### DETAILED DESCRIPTION OF THE INVENTION

Diapers for babies may include features to enhance the comfort and well-being of the baby. An example of a baby diaper is shown in FIG. 1, which includes a main body with one or more layers, for example a tri-layer structure, and a waistband or waist fastener at both ends and seals around the legs of the baby. In the example of a tri-layer structure, one may find a liquid-impermeable outer shell, a liquid absorbing and retaining central layer, and a liquid-permeable inner lining. The waistband may be composed of an elastic or a non-elastic band with an anchor at each end that is typically made of Velcro. The waistband may aide in keeping the diaper in position and it may act as a barrier between the inside and the outside of the diaper.

When a baby wears a diaper, for example a diaper of the general make-up described above, the diaper will hug the baby, especially through the diaper's waistband and the seals around the legs. The waistband and the leg seals may act as leak prevention, but the quality of leak prevention depends on the design and fit of the waistband and leg seals. Known diapers offer only insufficient leak protection, especially around the waistband and most particularly in places where the contact of the waistband with the baby is weak at times and thereby allowing leakage.

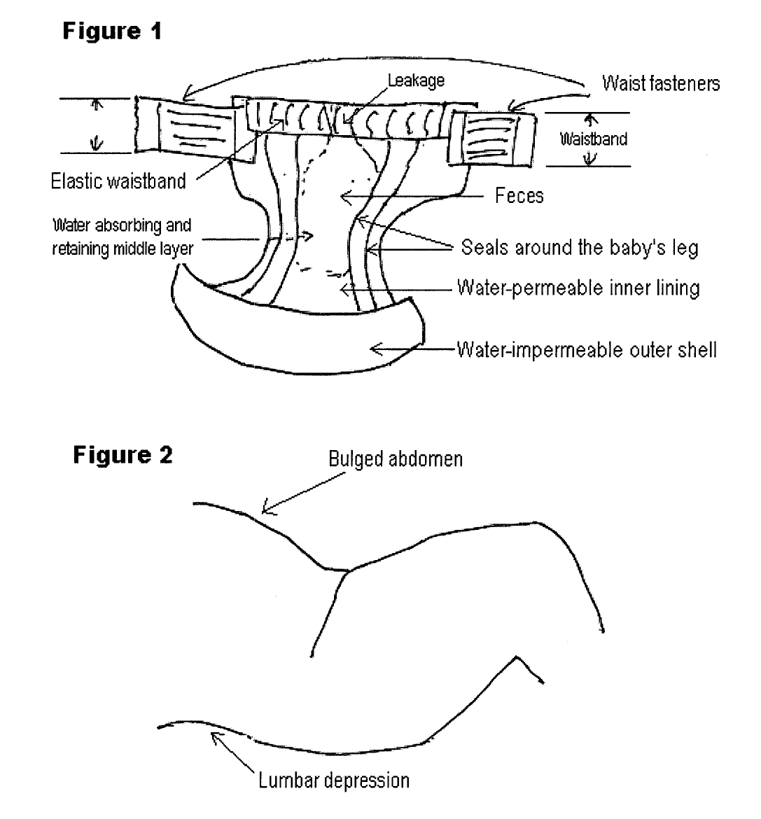

Leakage from a diaper depends on the design of the diaper and places where diaper leakage may occur can be examined by considering the baby's anatomy. Babies have a bulged abdomen and a slight dip in the lumbar region of the back. The waist of a baby is typically shaped like a cocktail glass on the abdominal side, with a larger dimension at the waist which tapes down towards the hip and pelvis (FIG. 2). Leak prevention in the front and back of the baby are particularly difficult, while in contrast the strong curvature around the hips results in a better seal through the waistband and therefore better leak prevention. The backbone of the baby dips around the lumbar region and creates a slightly uneven region at the backside around the spine. This unevenness of the spinal region makes a useful seal around the waist particularly difficult to achieve. The spinal region on the backside of the diaper is an area where diaper leaks likely occur (FIG. 1).

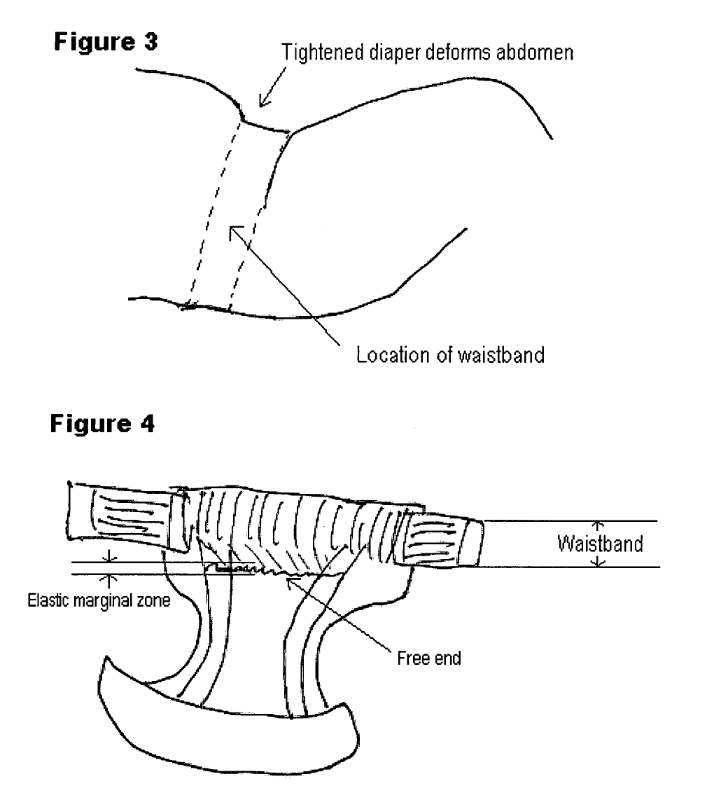

Leakage is particularly prone to occur when a diaper slips down, even to a minor extent, because it often loosens the leak barrier around the waist. The waistband of a diaper does not provide adequate leak prevention over time, especially after the baby has moved or was handled by an adult. In order to prevent a diaper accident, parents may have to resort to excessive tightening of the waist fastener (FIG. 3), which may result in discomfort to the baby or even harmful influence to the normal development of the baby.

Leak Guards of the Invention Enhance Leak Prevention of Diapers.

A diaper according to the current invention provides improved leak prevention. Diapers according to the invention provide in certain embodiments improved leak prevention around the waist of the baby, and, in other certain embodiments, on the front and backside of the baby or only on the front or only on the backside of the baby. In certain embodiments, a diaper according to the invention comprises a leak-proof guard capable of providing improved leak prevention. A leak-proof guard according to certain embodiments of the invention provides added leak protection around the waist of the baby and preferably on the front and/or backside of the baby and most preferably on the backside of the baby. In certain preferred embodiments, the leak-proof guard of the invention improves leak prevention in the area of the spinal column of the baby.

A leak-proof guard of the invention, in certain embodiments, comprises material inserted into, attached to and/or put into a diaper. In certain other embodiments, a leak-proof guard of the invention comprises material of the diaper that was reshaped, reworked and/or remade into a leak-proof guard of the invention. In yet certain other embodiments, a leak-proof guard of the invention comprises some combination of material that is inserted into, attached to and/or put into a diaper and material of the diaper that was reshaped, reworked and/or remade to result in a leak-proof guard of the invention. Material can readily be inserted into, attached to and/or put into a diaper, as realized by a skilled artisan reviewing the teachings herein, for example, by sewing, gluing, stitching, taping, clipping, buttoning, using Velcro, binding, or any other means that would result in the material as a, or part of a, leak-proof guard of the invention. Material of the diaper can also readily be reshaped, reworked and/or remade into a leak-proof guard of the invention, as realized by a skilled artisan reviewing the teachings herein, for example, by sewing, gluing, stitching, taping, clipping, buttoning, using Velcro, binding, or any other means that would result in the material of the diaper as a, or part of a, leak-proof guard of the invention.

The Location of the Leak Guards of the Invention.

A leak-proof guard of the invention, in certain embodiments, is located, when the diaper is worn by a baby, above the area in which the baby would excrete fluids and other matter into the diaper but below the upper edge of the diaper. In certain other embodiments, the leak-proof guard is located below the waistband of the baby. Where a diaper, in certain embodiments, has an area that functions as a waistband, then the leak-proof guard of the invention would preferably be located below that waistband. A waistband area of a diaper is typically found somewhere around or above the hips and typically not far from, or even very close to, the belly button level of the baby. An area may function as a waistband, for example, because the area fits tighter to the baby's skin when the diaper is worn as compared to the body of the diaper, or, for example, the waistband area may fit tightly in a way that is comparable to the seals around the baby's legs, or, for example, the waistband area may comprise some form of barrier to fluids and matter leaking from the diaper, or all of the above may apply to the waistband area. A waistband area may also be referred to as a functional waistband or simply as the waistband.

The leak-proof guard of the invention, in certain embodiments, gives added leak protection to the diaper, and preferably to other leak barriers of the diaper, so that it is preferably located to facilitate such added leak protection. For example, where a diaper has a waistband, the leak-proof guard of the invention is located, in certain embodiments, on the waistband or overlapping with the waistband, for example, the leak-proof guard and the waistband may overlap by 80 or about 80 percent or at least that amount, by 60 or about 60 percent or at least that amount, by 40 or about 40 percent or at least that amount, or by 20 or about 20 percent or at least that amount, or the overlap may be from 20 to 80 percent, or 20 to 60 percent, or 40 to 60 percent. In certain other embodiments, the leak-proof guard is located below the waistband. For example, the leak-proof guard of the invention is placed, in certain embodiments, so that it can exert a sufficient amount of pressure, or seal, to the baby's skin so that a desired leak prevention is achieved, but without unduly interfering with the baby's health and/or comfort. In certain embodiments, the leak-proof guard of the invention is located next to, or about next to, the waistband of the diaper, or, at a distance of 5 or about 5 millimeters from the waistband or at least that amount, or 10 or about 10 millimeters or at least that amount, or 15 or about 15 millimeters, or 20 or about 20 millimeters, or 25 or about 25 millimeters, or 30 or about 30 millimeters, or 0 to 30 millimeters, or 2 to 20 millimeters, or 5 to 30 millimeters, or 5 to 20 millimeters, or 10 to 30 millimeters, or 10 to 20 millimeters. The preferred placing of the leak-proof guard of the invention with regards to the waistband of the diaper is such that one achieves desired leak prevention without unduly interfering with the health and/or comfort of the baby.

The Dimension of Leak Guards of the Invention.

A leak-proof guard of the invention, in certain embodiments, is long enough to fit around the waist of the baby, in certain other embodiments, it covers a fraction of the waistline of the baby, for example, 5 or about 5 percent of the waistline or at least that amount, or 10 or about 10 percent of the waistline or at least that amount, or 15 or about 15 percent of the waistline or at least that amount, or 20 or about 20 percent of the waistline or at least that amount, or 30 or about 30 percent of the waistline or at least that amount, or 40 or about 40 percent of the waistline or at least that amount, or 50 or about 50 percent of the waistline or at least that amount, or 5 to 50 percent of the waistline, or 5 to 30 percent of the waistline, or 10 to 50 percent of the waistline, or 10 to 30 percent of the waistline, or 10 to 20 percent of the waistline, or 20 to 30 percent of the waistline. These percentage values for the fraction of the waistline covered by the leak-proof guard of the invention, in certain embodiments, refer to the percentage covered by a single leak-proof guard or by two, three, four or more leak-proof guards combined.

The width of a leak-proof guard of the invention can be measured as the extension of the leak-proof guard along an axis perpendicular to the waistline of the baby and parallel to the baby's skin. In certain embodiments, the width of the leak-proof guard is uniform or substantially uniform, while in certain other embodiments, the width of the leak-proof guard may vary. In certain embodiments, the width of the leak-proof guard is sufficient to improve leak prevention of the diaper but not so much as to unduly interfere with the comfort and/or health of the baby. In certain embodiments, the width of the leak-proof guard is 2 or about 2 millimeters or at least that amount, or 5 or about 5 millimeters or at least that amount, or 10 or about 10 millimeters or at least that amount, or 15 or about 15 millimeters or at least that amount, or 20 or about 20 millimeters or at least that amount, or 30 or about 30 millimeters or at least that amount, or 40 or about 40 millimeters or at least that amount, or 50 or about 50 millimeters or at least that amount, or 60 or about 60 millimeters or at least that amount, or 80 or about 80 millimeters or at least that amount, or 100 or about 100 millimeters or at least that amount, or 2 to 100 millimeters, or 2 to 50 millimeters, or 5 to 50 millimeters, or 10 to 40 millimeters, or 10 to 30 millimeters, or 20 to 40 millimeters. A leak-proof guard with varying width, in certain embodiments, has a minimum width and a maximum width. The minimum width of the leak-proof guard, in certain embodiments, is sufficient to achieve a desired improvement in leak-prevention of the diaper. In certain embodiments of the invention, the leak-proof guard has a maximum width that is larger than the minimum extension, for example, 10 or about 10 percent larger or at least that amount, or 20 or about 20 percent larger or at least that amount, or 40 or about 40 percent larger or at least that amount, or 60 or about 60 percent larger or at least that amount, or 80 or about 80 percent larger or at least that amount, or 10 to 80 percent larger, or 20 to 60 percent larger.

The Shapes and Orientations of Leak Guards of the Invention.

A leak-proof guard according to certain embodiments of the invention comprises a band, a pad, a cushion, a pillow, a support, an absorber, a roll, or other device capable of making the diaper less likely to leak when compared to a diaper without the leak-proof guard. A leak-proof guard of the invention may, in certain embodiments, be comprised of a single band, pad, cushion, pillow, support, absorber, roll, or other device, or it may be comprised of more than one band, pad, cushion, pillow, support, absorber, roll, or other device, for example, 2, 3, 4, 5, 6, 7, 8, 9, 10, or more, or 1-10, or 2-8, or 2-4, or 4-6, or 1-7, or 1-5, or 3-5. The leak-proof guard of the invention, in certain embodiments, may be in the shape of a band, a line, a dot, a square, a rectangle, a circle, an arc, a diamond, a trapezoid, or any other shape that can be arranged in the diaper so that the leak-proof properties of the diaper are improved. The orientation of the leak-proof guard of the invention in the diaper, in certain embodiments, can be parallel to the waist line of the diaper, or at an angle to the waist line, for example, a steep angle, or a shallow angle, or an angle of 5 or about 5 degrees or at least that amount, or 10 or about 10 degrees or at least that amount, or 20 or about 20 degrees or at least that amount, or 30 or about 30 degrees or at least that amount, or 45 or about 45 degrees or at least that amount, or 5 to 45 degrees, or 5 to 20 degrees, or 10 to 30 degrees, when measured between the waist line of the diaper and a longitudinal axis or longer major axis of the leak-proof guard, if one exists.

The Material and Thickness of Leak Guards of the Invention.

A leak-proof guard of the invention, in certain embodiments, can be made of different materials, for example, gauze, cloth, paper, sponge, plastic, or any other material that is capable of improving leak prevention and that does not unduly interfere with the baby's comfort and/or health. A leak-proof guard of the invention, in certain embodiments, can be made of non-perforated, perforated or semi-perforated material. The thickness of the leak-proof guard can be measured as the extension of the leak-proof guard of the invention along an axis perpendicular to the skin of the baby. The thickness of the leak-proof guard of the invention, in certain embodiments, is measured when the diaper is not worn by anyone. In certain other embodiments, the thickness of the leak-proof guard is measured when the guard is not subject to pressure, or when the diaper is worn by a baby. In certain embodiments, the thickness of the leak-proof guard is 1 or about 1 millimeter or at least that amount, or 2 or about 2 millimeters or at least that amount, or 3 or about 3 millimeters or at least that amount, or 4 or about 4 millimeters or at least that amount, or 5 or about 5 millimeters or at least that amount, or 7 or about 7 millimeters or at least that amount, or 10 or about 10 millimeters or at least that amount, or 15 or about 15 millimeters or at least that amount, or 20 or about 20 millimeters or at least that amount, or 30 or about 30 millimeters or at least that amount, or 50 or about 50 millimeters or at least that amount, or 1 to 50 millimeters, or 2 to 30 millimeters, or 3 to 20 millimeters, or 4 to 15 millimeters, or 5 to 10 millimeters, or 5 to 15 millimeters or any other thickness that improves the leak prevention of the diaper but does not unduly interfere with the comfort and/or health of the baby. In certain embodiments, the thickness of the leak-proof guard is uniform or substantially uniform. In certain other embodiments, the thickness is not uniform but has a maximum thickness and a minimum thickness. In certain embodiments of the invention, the leak-proof guard has a maximum thickness that is larger than the minimum thickness, for example, by 10 or about 10 percent or at least that amount, or by 20 or about 20 percent or at least that amount, or by 40 or about 40 percent or at least that amount, or by 60 or about 60 percent or at least that amount, or by 80 or about 80 percent or at least that amount, or by 10 to 80 percent, or by 20 to 60 percent. In certain preferred embodiments, the thickness of the leak-proof guard is designed to accommodate for the baby's bodily contours so that the leak-prevention properties of the diaper are most suitably enhanced. For example, the leak-proof guard's thickness, in certain preferred embodiments, is designed to provide even or about even contact with the baby's skin around the spinal cord region, or the belly button region, or some other region of interest. For example, by enhancing the thickness of the leak-proof guard to account for the lumbar depression of the baby, and preferably with the thickness tapering off to the sides of the baby's body, in certain embodiments, a preferred leak-prevention property of the leak-proof guard of the invention is accomplished.

The Sealing Zone Leak Guard of the Invention.

In certain other embodiments, a leak-proof guard of the invention comprises a sealing zone. The sealing zone, in certain embodiments, is located below the waistband of the diaper and preferably close enough to the waist line of the baby to effectively improve leak prevention of the diaper. The sealing zone, in certain embodiments, may comprise material that is inserted into, attached to and/or put into the diaper so that the sealing zone overlaps with the waistband, while in certain other embodiments the sealing zone is located below the waistband at no distance or substantially no distance, or at a distance of 2 or about 2 millimeters or at least that amount, 5 or about 5 millimeters or at least that amount, or 10 or about 10 millimeters or at least that amount, or 20 or about 20 millimeters or at least that amount, or 30 or about 30 millimeters or at least that amount, or 50 or about 50 millimeters or at least that amount, or 0 to 30 millimeters, or 2 to 20 millimeters, or 5 to 20 millimeters, or 2 to 10 millimeters. In certain embodiments, the sealing zone is separate from the waistband, for example, so that stretching the waistband does not affect the shape or stretching of the sealing zone, or not much, or, for example, so that the sealing properties of the sealing zone are independent from or not unduly affected by the fit or stretching of the waistband.

In certain embodiments, the sealing zone guard is attached to the main body of the diaper along the upper edge of the sealing zone and in certain preferred embodiments it is attached along the upper edge and along the side edges of the sealing zone guard to the main body of the diaper. In certain embodiment, the sealing zone guard is attached along each of its edges in its entirety to the main body of the diaper or it may be attached along parts of the edges. In certain embodiments, the sealing zone guard is attached along its entire upper edge but only along parts of the side edges.

The sealing zone, in certain embodiments, is comprised of material on the inside of the diaper which is attached to the body of the diaper at one end but not at the other end. In certain preferred embodiments, the attached end is the end that is closer to the waist line of the baby and the unattached end is the end opposite to the attached end and farther away from the waist line of the baby. In certain preferred embodiments, the material of the sealing zone forms a space between the unattached end of the material and the body of the diaper. In certain other embodiments, the unattached end of the sealing zone is prone to extending or reaching away from the diaper's body and therefore towards the baby wearing the diaper. In certain preferred embodiments, the unattached end of the sealing zone forms a seal with the skin of the baby wearing the diaper.

The shape of the sealing zone, in certain embodiments, may be a band, a line, a dot, a square, a rectangle, a circle, an arc, a diamond, a trapezoid, or any other shape that can be arranged in the diaper so that the leak prevention properties of the diaper are improved without unduly interfering with the health and/or comfort of the baby. The size of the sealing zone can be varied. In certain embodiments, the maximum extension of the sealing zone is 5 or about 5 millimeters or at least that amount, or 10 or about 10 millimeters or at least that amount, or 20 or about 20 millimeters or at least that amount, or 30 or about 30 millimeters or at least that amount, or 50 or about 50 millimeters or at least that amount, or 70 or about 70 millimeters or at least that amount, or 100 or about 100 millimeters or at least that amount, or 150 or about 150 millimeters or at least that amount, or 200 or about 200 millimeters or at least that amount, or 5 to 200 millimeters, or 5 to 150 millimeters, or 5 to 100 millimeters, or 10 to 100 millimeters, or 20 to 100 millimeters, or 30 to 100 millimeters, or 40 to 100 millimeters, or 5 to 50 millimeters, or 10 to 50 millimeters, when measured along the waist line of the baby, or a line parallel thereto.

In certain other embodiments, the maximum extension of the sealing zone is 5 or about 5 millimeters or at least that amount, or 10 or about 10 millimeters or at least that amount, or 20 or about 20 millimeters or at least that amount, or 30 or about 30 millimeters or at least that amount, or 50 or about 50 millimeters or at least that amount, or 70 or about 70 millimeters or at least that amount, or 100 or about 100 millimeters or at least that amount, or 150 or about 150 millimeters or at least that amount, or 200 or about 200 millimeters or at least that amount, or 5 to 200 millimeters, or 5 to 100 millimeters, or 10 to 70 millimeters, or 20 to 50 millimeters, or 10 to 30 millimeters, when measured perpendicular to the waist line and parallel to the baby's skin, when the diaper is worn. These measurements of the sealing zone, in certain embodiments, represent the maximum extension of the sealing zone along each axis. The size of the sealing zone, in certain preferred embodiments, is uniform or substantially uniform when measure along one or both of the two axes discussed above (i.e., the axis parallel and the axis perpendicular to the waist line), however, in certain other embodiments, the measurements can be varied; thus resulting in different shapes of the sealing zone. The minimum extension of the sealing zone along the two axes, in certain embodiments, is a fraction of the maximum extension, for example, 10 or about 10 percent thereof or at least that amount, or 20 or about 20 percent thereof or at least that amount, or 40 or about 40 percent thereof or at least that amount, or 60 or about 60 percent thereof or at least that amount, or 80 or about 80 percent thereof or at least that amount, or 10 to 80 percent, or 20 to 60 percent, and it can be the same or different along each of the two axes.

In certain preferred embodiments, the sealing zone comprises a rectangle, including a perfect rectangle or a rough or approximate rectangle, that is long enough to fit around the baby and is about one to about three centimeters wide. In certain other preferred embodiments, the sealing zone is rectangular and long enough to fit around 5 or about 5 percent of the waistline or at least that amount, or 10 or about 10 percent of the waistline or at least that amount, or 15 or about 15 percent of the waistline or at least that amount, or 20 or about 20 percent of the waistline or at least that amount, or 30 or about 30 percent of the waistline or at least that amount, or 40 or about 40 percent of the waistline or at least that amount, or 50 or about 50 percent of the waistline or at least that amount, or 5 to 50 percent of the waistline, or 5 to 30 percent of the waistline, or 10 to 50 percent of the waistline, or 10 to 30 percent of the waistline, or 10 to 20 percent of the waistline, or 20 to 30 percent of the waistline. In certain preferred embodiments, the rectangular sealing zone is located on the front and/or back side of the baby where it may cover any one of the above percentages on the front and/or backside of the baby. In certain embodiments, the width of the rectangular sealing zone is 5 or about 5 millimeters or at least that amount, or 10 or about 10 millimeters or at least that amount, or 20 or about 20 millimeters or at least that amount, or 30 or about 30 millimeters or at least that amount, or 50 or about 50 millimeters or at least that amount, or 70 or about 70 millimeters or at least that amount, or 100 or about 100 millimeters or at least that amount, or 150 or about 150 millimeters or at least that amount, or 200 or about 200 millimeters or at least that amount, or 5 to 200 millimeters, or 5 to 100 millimeters, or 10 to 70 millimeters, or 20 to 50 millimeters, or 10 to 30 millimeters.

In certain embodiments, the sealing zone guard of the invention comprises elastic material at the bottom and non-elastic material at the top. In certain preferred embodiments, the elastic material comprises 1 or about 1 percent or at least that amount, 5 or about 5 percent or at least that amount, 10 or about 10 percent or at least that amount, or 20 or about 20 percent or at least that amount, or 30 or about 30 percent or at least that amount, or 40 or about 40 percent or at least that amount, or 60 or about 60 percent or at least that amount, or 80 or about 80 percent or at least that amount, or 1 to 90 percent, or 5 to 80 percent, or 10 to 80 percent, or 20 to 60 percent, or 10 to 40 percent, or 20 to 30 percent of the sealing zone, with the remainder of the sealing zone being comprised of non-elastic material.

The thickness of the material of the sealing zone comprises any thickness sufficient to improve leak prevention of the diaper and, in certain embodiments, comprises a medium or median or average thickness of 1 or about 1 millimeter or at least that amount, or 2 or about 2 millimeters or at least that amount, or 3 or about 3 millimeters or at least that amount, or 4 or about 4 millimeters or at least that amount, or 5 or about 5 millimeters or at least that amount, or 7 or about 7 millimeters or at least that amount, or 10 or about 10 millimeters or at least that amount, or 15 or about 15 millimeters or at least that amount, or 20 or about 20 millimeters or at least that amount, or 1 to 20 millimeters, or 2 to 10 millimeters, or 1 to 10 millimeters, or 3 to 7 millimeters, or any other thickness that improves the leak prevention of the diaper but does not unduly interfere with the comfort and/or health of the baby. While in certain preferred embodiments, the thickness of the sealing zone material is uniform or substantially uniform, the thickness may be varied in any way. For example, the sealing zone material may be thicker at the attached end or at the unattached end or in the section of the sealing zone that is located at or around the area of the backbone and/or the belly button of the baby and may include the front side and/or the back side of the baby.

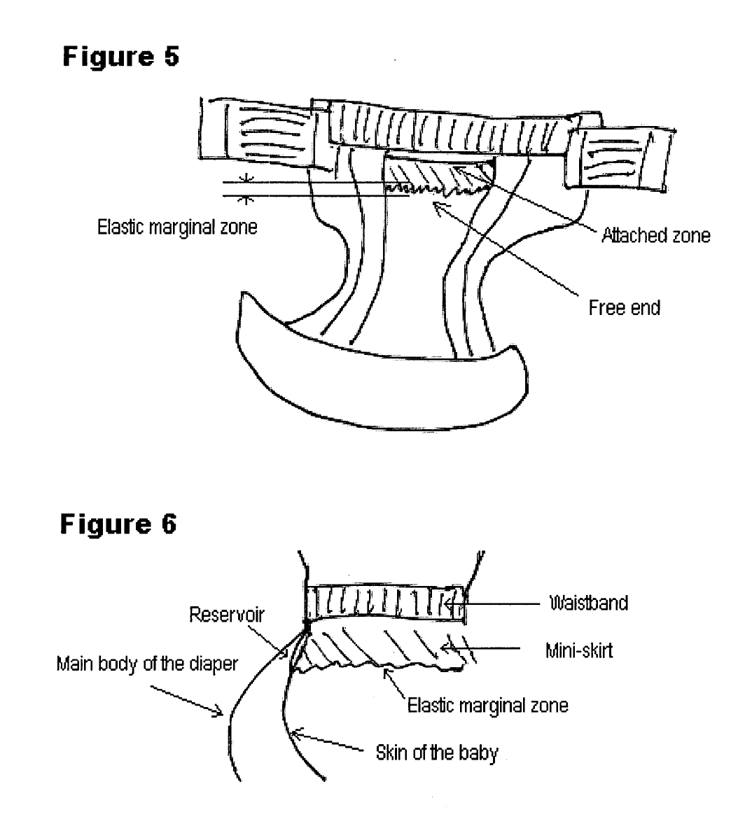

In certain embodiments, the sealing zone leak-proof guard of the invention is like a mini-skirt that is attached to the main body of diaper. The mini-skirt may, in certain embodiments, form a partial or completely circumferential fit around the baby. In certain preferred embodiments, the mini-skirt forms a partial fit in the back of the baby, and more preferably in the backbone area of the baby. The mini-skirt, in certain embodiments, is a downward extension of the waistband with an elastic marginal zone (or marginal zone) on its free lower end (FIG. 4), or a separate strip of a band bonded or attached to the main body of the diaper preferably with an elastic marginal zone on its free end (FIG. 5).

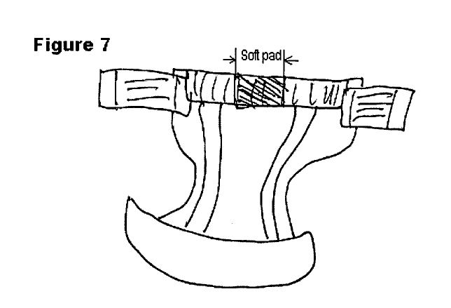

In order to form an improved skin seal, one or more mini-skirts are positioned at the area above the beginning of the gluteal cleavage that will provide an extra transversal barrier in addition to the transversal barrier provided by the waistband or waist fastener. In order to provide comfort to the diaper wearer and perform more effective barrier function, the mini-skirt is preferably placed below the transversal barrier created by a waistband or waist fastener. The free end of the mini-skirt has an elastic property at its marginal zone, which helps to form a seal with the skin of the baby and provides improved leak-proof features to the diaper. The elasticity of the transversal barrier at its marginal zone is a preferred embodiment of the invention. Due to the elastic nature at its marginal zone in certain preferred embodiments, the length of the free end of the mini-skirt is shorter than that of the attached end in a relaxed state. This will preferably result in slight folding or bending of the sealing guard, in certain embodiments, towards the baby's skin and preferably when the diaper is worn. Therefore, in certain embodiments, the snuggly fit sealing zone guard and the loose fit main body of the diaper create a reservoir for temporary storage of the moving feces during the bowl movement and reduce the incident of diaper accident (FIG. 6).

The free end of the mini-skirt forms a suitable seal with the skin of the baby under varying conditions of movement or rest of the baby. In certain embodiments, the free end of mini-skirt has an elastic marginal zone. In one embodiment, the elastic marginal zone is attached to the free end of mini-skirt on both sides. In another embodiment, the elastic marginal zone is attached to the free end of mini-skirt on one side on either facet. In yet another embodiment, the elastic marginal zone is embedded in the fabric of the mini-skirt.

In a preferred embodiment, the main body of the mini-skirt is a sheet or film of material without elastic components outside of marginal zone at free end. In another embodiment, the mini-skirt is a sheet of material with some elastic components outside of the marginal zone. In a preferred embodiment, the elastic marginal zone is located at the margin of the free end of mini-skirt. But the marginal zone can be located off the margin of the free end to perform a similar transversal barrier function.

The fabric of the mini-skirt or the sealing zone, in certain embodiments, is a sheet of woven material, such as a sheet of cloth, or a sheet of molded material, such as a sheet of plastic, or a sheet of non-woven material, such as a sheet of paper, or a sheet of any combination of materials from woven, molded, and non-woven materials. The fabric of the mini-skirt or the sealing zone, in certain embodiments, is a sheet of material (such as paper and cloth) from a natural source (plants and/or animals), or a sheet of material (such as plastic and other polymeric product) that is synthetic and made through biological, chemical and/or physical methods, or a sheet of material made from a combination of natural and synthetic products.

In certain embodiments, the mini-skirt or the sealing zone is made of a liquid-impermeable fabric that will provide better waste retention capacity. Preferably, the liquid-impermeable fabric is a breathable material that will allow water vapor to escape and preferably minimize diaper rash and increase the comfort and/or health of the baby. In certain embodiments, the mini-skirt or the sealing zone comprises a perforated fabric that will facilitate water vapor escaping. In certain other embodiments, the mini-skirt or the sealing zone is a liquid-permeable fabric that will only provide a sufficient barrier during pressurized defecation to assist the barrier function provided by the barrier function established by the waistband.

In certain embodiments, the mini-skirt is positioned in the frontal (abdominal side) and/or back (lumbar spinal side) side of the diaper where it may provide leak protection as a transversal barrier for both sides of the diaper. In another embodiment, the mini-skirt is positioned only at the backside of the diaper, preferably in a centered arrangement, where it may provide an additional transversal barrier for the backside.

In certain embodiments, the mini-skirt is connected with the main body of the diaper on its upper side or edge and, in certain embodiments, with the leg seals to create a pocket. In another embodiment, one or both ends of the mini-skirt are attached to the main body of diaper as well. In yet another embodiment, one or both lateral ends are free from any attachment. In this embodiment, the mini-skirt does not form a pocket but only serves as a baffle to slow down the flow of fluids and other matter.

The Padded Waistband Leak Guard of the Invention.

In certain embodiments, the leak-proof guard of the invention comprises a waistband that incorporates a soft pad around the lumbar spinal region (FIG. 7). Upon compression by fastening of the waistband, the soft pad changes its shape to make a firmer or substantially firmer contact with the skin around the lumbar spinal region, preferably resulting in improved leak prevention and preferably regardless of variation in body contours of the baby. A firm skin contact of the soft pad improves the contact of the transversal barrier of the waistband and the skin around spinal region, and preferably enhances the barrier function of the waistband or waist fastener by filling the gap or channel around the spinal region to from a better seal in that region.

In certain embodiments, the soft pad is placed on the waistband as appendage that is placed between the waistband and the back skin of the baby. In certain other embodiments, the soft pad is embedded or integrated into the waistband. Preferably, the soft pad facilitates a firmer contact between waistband and the skin of the baby around the lumbar spinal region and preferably results in improved leak prevention. The soft pad can be shaped in various geometric forms, including but not limited to a square, a rectangle, an oval, a diamond, a trapezoid, a circle, an arc, or any irregular shapes. The length of the soft pad can be the entire length of the waistband or a fraction of the waistband, for example, 5 or about 5 percent or at least that amount, or 10 or about 10 percent or at least that amount, or 15 or about 15 percent or at least that amount, or 20 or about 20 percent or at least that amount, or 30 or about 30 percent or at least that amount, or 40 or about 40 percent or at least that amount, or 50 or about 50 percent or at least that amount, or 5 to 50 percent, or 5 to 30 percent, or 5 to 20 percent, or 5 to 10 percent, or 10 to 20 percent.

The width of the soft pad may be, in certain embodiments, is 5 or about 5 millimeters or at least that amount, or 10 or about 10 millimeters or at least that amount, or 20 or about 20 millimeters or at least that amount, or 30 or about 30 millimeters or at least that amount, or 50 or about 50 millimeters or at least that amount, or 70 or about 70 millimeters or at least that amount, or 100 or about 100 millimeters or at least that amount, or 5 to 100 millimeters, or 10 to 50 millimeters, or 20 to 30 millimeters, or 10 to 20 millimeters. In certain preferred embodiments, the soft pad is sufficiently thick to form a firm contact between the waistband and the skin of diaper wearer around the lumbar spinal region, for example, 2 or about 2 millimeters or at least that amount, or 4 or about 4 millimeters or at least that amount, or 6 or about 6 millimeters or at least that amount, or 10 or about 10 millimeters or at least that amount, or 15 or about 15 millimeters or at least that amount, or 20 or about 20 millimeters or at least that amount, or 1 to 20 millimeters, or 2 to 10 millimeters, or 1 to 10 millimeters, or 3 to 7 millimeters, or any other thickness that improves the leak prevention of the diaper but does not unduly interfere with the comfort and/or health of the baby. In certain embodiments, the thickness of the soft pad is designed to accommodate for the baby's bodily contours so that the leak-prevention properties of the diaper are most suitably enhanced. For example, the soft pad, in certain preferred embodiments, is designed to provide even or about even contact with the baby's skin around the spinal cord region. For example, by enhancing the thickness of the soft pad to account for the lumbar depression of the baby, and preferably with the thickness tapering off to the sides of the baby's body, in certain embodiments, a preferred leak-prevention property of the soft pad of the invention is accomplished.

In one embodiment, the soft pad is made of nature products, including but not limited to cotton, rubber and sponge. In another embodiment, the soft pad is made of synthetic materials, including but not limited to flexible plastic foam, silicone rubber and fiberglass fibers. In yet another embodiment, the soft pad is made of a combination of nature and synthetic materials. In one embodiment, the soft pad is made of soft but solid material. In another embodiment, the soft pad is made of gel or liquid in a sealed container that has soft skin. In yet another embodiment, the soft pad is made of a combination of soft padding material and material capable of absorbing water.

In certain embodiments, the soft pad is made of a cover material shell and a soft padding material core. In certain embodiments, the cover material shell is made of a liquid-impermeable fabric. In other embodiments, the cover material shell is made of a liquid-permeable fabric. In yet another embodiment, the cover material shell is made of a combination of a liquid-impermeable and a liquid-permeable fabric, with the liquid-permeable fabric positioned at closer proximity to the anus.

A leak-proof guard of the invention will enhance the leak prevention properties of a diaper, and reduce the incident of diaper accident associated with leakage, especially on the front and/or backside of the baby. In certain embodiments, more than one leak-proof guard of the invention is used in a diaper, for example, 2, 3, 4, 5, 6, 7, 8, 9, 10, or more leak-proof guards, or 1-10, or 2-8, or 2-4, or 4-6, or 1-7, or 1-5, or 3-5 leak-proof guards of the invention are used in a diaper. A leak-proof guard of the invention can be designed using features of the different embodiments described herein and applying those features to a leak-proof guard. When more than one leak-proof guard is used in a diaper, the leak-proof guards may be designed in the same way or in different ways, for example, a diaper may include different leak-proof guards designed according to different embodiments described herein and the different leak-proof guards may be different from one another.

The present invention is further illustrated by the following examples, which are not intended to be limiting in any way whatsoever.

### EXAMPLES

The following examples illustrate diapers with a leak-proof guard according to certain embodiments of the invention and demonstrate that these diapers provide improved leak prevention. Moreover, these examples illustrate that the diapers that were tested improved leak prevention in areas that are particular prone to leaking, for example, the backside of the baby around the spinal region. Diapers used in these examples were made by using material from a commercially available diaper (a size three Huggies diaper) but material from other commercially available diapers can also be used to make a diaper of the design used in these examples (for examples, Pampers and Tyco International diapers). Materials used in making the diapers tested were the back sheet and top sheet of the commercially available diaper. The back sheet is a water impermeable and breathable cloth-like sheet and it is suitable for construction of a leak-proof barrier in particular where the barrier makes contact with the skin of the baby. Elastic rubber bands or soft foam material (flexible polyurethane foam) were used for establishing a snug seal between leak-proof barrier and baby's skin. The rubber bands used were about 1 mm thick and about 2 mm wide and were obtained from a drug store (Walgreens). The soft foam was obtained from material used as filling material for shipping packages. Three designs of diapers were prepared in accordance with certain embodiments of the invention for testing their leak-prevention properties.

### Example 1

### Mini-Skirt with Elastic Marginal Zone

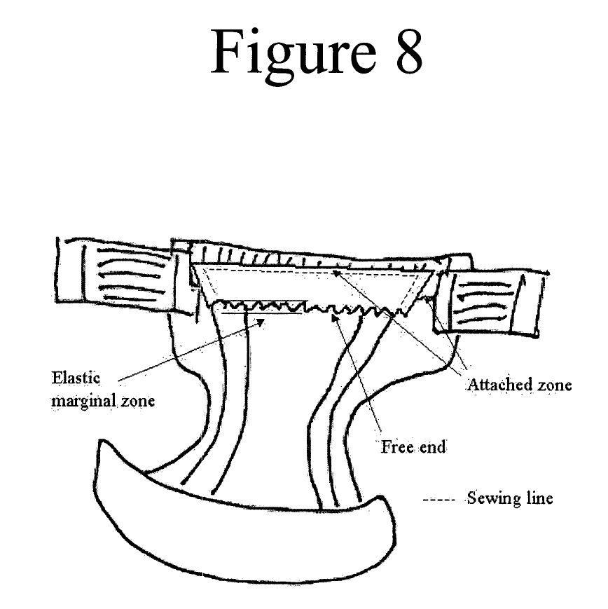

A mini-skirt leak-proof guard was made by attaching a rectangular shaped (about 12 cm×3.5 cm) back sheet of the diaper to the main body of the diaper (FIG. 8). It was sewed to the diaper at the top and at the two sides; hence, the mini-skirt and the main body of the diaper formed a pocket with a downward pointing open or free end. The sides were sewn to the diaper at a slanted angle of about 30 degrees inward. The elastic band was sewn into the botton edge of the mini-skirt so that the band was extended but not stretched but would be stretched once the diaper is worn by a baby. This way, the bottom edge of the mini-skirt would remain stretched and thereby maintain a seal with the baby's skin. The slanted sides also make the free end slightly shorter than the attached top side when the diaper is fully stretched. This arrangement therefore helps to maintain the elasticity of the free end when it is fully stretched.

The position of the free end of the mini-skirt, or the opening of the pocket, results from the fact that the elastic zone (see below on how the elastic zone was created) will make skin contact above the gluteal cleavage. The free end of the mini-skirt was placed around an imaginary line about 1.5 cm above the lower border of the waistband fastener (FIG. 8). The free end of mini-skirt has an elastic zone on its margin to form a snug seal against baby's skin. The elastic marginal zone will be stretched once the diaper is properly placed on the baby. The stretched elastic marginal zone establishes a snug seal around the waistband independent of the body position of baby.

The marginal elastic zone was constructed by folding the back sheet into the tunnel at the free end. A stretched elastic rubber band (about 6 to 7 cm long before stretching) was placed in the tunnel with both ends attached to the main body of diaper. The length and elasticity of the elastic rubber band was selected based on two criteria, first, having sufficient tension to form a snug seal once stretched; and second, that it does not result in discomfort to the baby.

Ten diapers as described in this example were tested on a five-month old baby. In no instance was any leakage of any kind across the leak-proof barrier according to the invention observed. No leakage occurred on the baby's backside. In many instances was feces observed in the pocket formed by the mini-skirt and the main body of the diaper. The leak-proof guard tested in this example therefore prevented leakage in all tests and never allowed leakage to occur.

### Example 2

### Mini-Skirt with Soft Foam Insert (Flexible Polyurethane Foam)

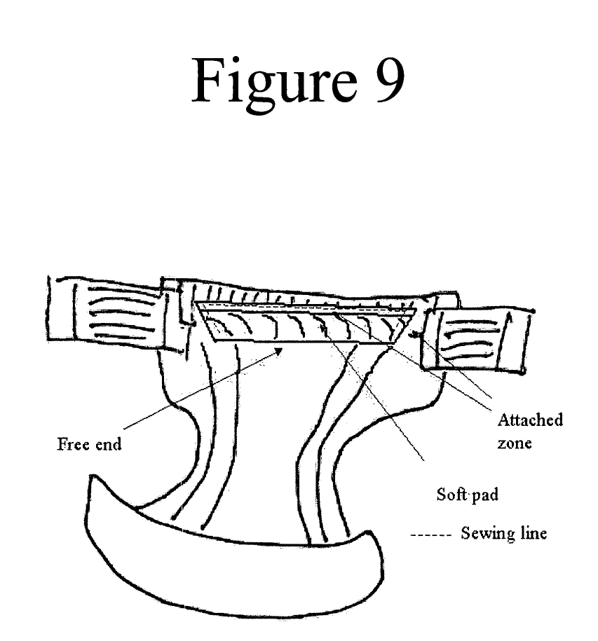

A mini-skirt was made of an attached tube (12 cm×4.5 cm without soft foam insert) made from back sheet material that was attached to the main body of the diaper at the top and the sides (FIG. 9). The sides were sewn to the diaper at a slanted angle of about 15 degrees inward. This makes the free end of mini-skirt slightly shorter than the attached top when the diaper is fully stretched. This arrangement helps to keep the free end open. The lower part of the mini-skirt was filled with a soft foam insert to result in a tube shape (about 10 cm×1.5 cm×1.5 cm rod shape, but which may be cylindrical or prism shape with similar dimensions). The free end of mini-skirt was located approximately on a level of about 1.5 cm above the lower border of the waistband fasteners (FIG. 9). The position of the free end of the mini-skirt was determined so that the soft foam insert makes contact with the baby's skin above the gluteal cleavage. The soft foam insert was deformed once the diaper was put on the baby; therefore, a snug seal was established regardless of the baby's body position. The space between the free end of the mini-skirt and the main body of the diaper provided a reservoir for feces during a large bowl movement.

More than thirty diapers as described in this example with a mini-skirt with a soft foam insert were tested on a five or six month old baby. In no instance was any leakage across the leak-proof guard of the invention observed. No leakage occurred on the backside of the baby. In many instances was feces observed in the space formed by the mini-skirt guard and the main body of the diaper. The leak-proof guard tested in this example therefore prevented leakage in all tests.

### Example 3

### Soft Pad Type of Leak-Proof Barrier

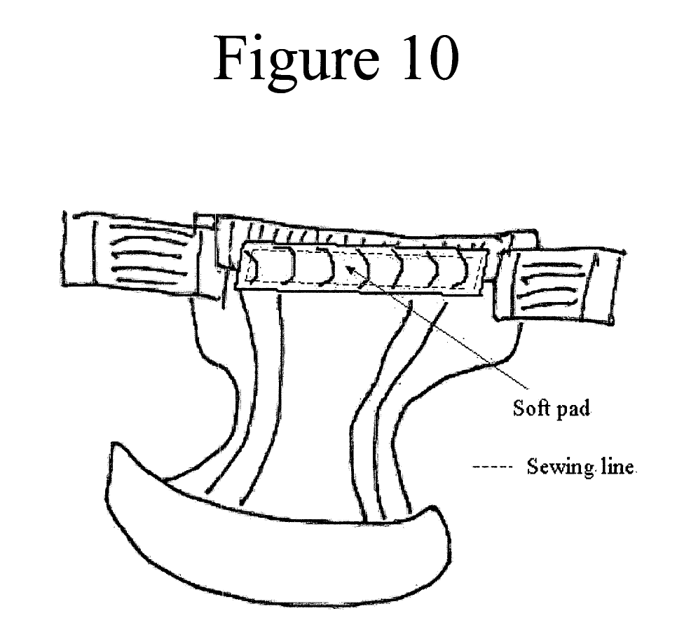

A soft pad type of leak-proof barrier was tested that was similar to a mini-skirt with soft foam insert except that all sides of the mini-skirt were attached to the main body of the diaper (FIG. 10). A tube was formed by sewing a back sheet (12 cm×5 cm) onto the main body of the diaper at the top, at the sides and at the lower side with about 3 cm spacing between the upper and the lower sewing line. The lower border of the tube was positioned approximately at an imaginary line about 1.5 cm above the lower end of the waistband fastener (FIG. 10). A soft pad was formed by inserting a soft foam insert (about 10 cm×1.5 cm×1.5 cm flexible polyurethane foam) inside the tube prior to attachment on all sides. The soft pad was deformed once the diaper was put on the baby; therefore, a snug seal was established regardless of the baby's position.

More than ten diapers as described in this example with soft pad were tested on a five to six month old baby. In no instance was any leakage across the leak-proof guard of the invention observed. No leakage occurred on the backside of the baby. In no instance, any of the modified diaper resulted in fecal leakage on the backside. The leak-proof guard tested in this example therefore prevented leakage in all tests.

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein, will become apparent to those skilled in the art from the foregoing description. Such modifications are intended to fall within the scope of the appended claims. All cited publications, patents, and patent applications are herein incorporated by reference in their entirety.

What is claimed is:

1. A diaper comprising a sealing zone leak-proof guard wherein said sealing zone comprises a seal that is capable of trapping a baby's waste between itself and the diaper's main body, wherein said sealing zone does not comprise the diaper's waistband and wherein said sealing zone is capable of improving the leak prevention properties of the diaper by providing a barrier to leakage from the diaper.

2. The diaper of claim 1, wherein said sealing zone comprises elastic material and non-elastic material.

3. The diaper of claim 1, wherein said sealing zone is located below the waistband of the diaper.

4. The diaper of claim 1, wherein said sealing zone overlaps with the waistband of the diaper.

5. The diaper of claim 1, wherein said sealing zone is rectangular.

6. The diaper of claim 1, wherein said sealing zone is trapezoid.

7. The diaper of claim 1, wherein said sealing zone is an arc.

8. The diaper of claim 1, wherein said sealing zone is located in the front of the baby.

9. The diaper of claim 1, wherein said sealing zone is located in the back of the baby.

10. The diaper of claim 1, wherein said sealing zone comprises 2 to 4 leak-proof guards.

11. A diaper comprising a padded waistband leak-proof guard wherein said leak-proof guard is located around the spinal region of the baby and wherein the leak-proof guard is capable of improving the leak prevention properties of the diaper around the spinal region.

12. The diaper of claim 11, wherein said padding is capable of adapting to the shape of a baby around the spinal region.

13. The diaper of claim 1 1, wherein said leak-proof guard overlaps with the waistband of the diaper.

14. The diaper of claim 11, wherein said leak-proof guard comprises 2 to 4 paddings.

15. A diaper comprising a leak-proof guard wherein said leak-proof guard provides a barrier to leakage from the diaper in the front and back of the baby around the baby's center line, but not around the hips of the baby and wherein said leak-proof guard does not comprise the diaper's waistband.

16. The diaper of claim 15, wherein said leak-proof guard is located below the waistband of the diaper.

17. The diaper of claim 15, wherein said sealing zone overlaps with the waistband of the diaper.

18. The diaper of claim 15, wherein said leak-proof guard is capable to adapting to the shape of a baby.

19. The diaper of claim 15, wherein said leak-proof guard is rectangular in shape.

20. The diaper of claim 15, wherein said leak-proof guard is trapezoid in shape.

21. The diaper of claim 15, wherein said leak-proof guard is comprised of 2 to 4 leak-proof guards.
